(19) United States
(12) Patent Application Publication
(10) Pub. No.: US 2018/0103857 A1
(43) Pub. Date: Apr. 19, 2018

(54) SENSOR FOR SENSING A BIOMETRIC FUNCTION

(71) Applicant: OSRAM Opto Semiconductors GmbH, Regensburg (DE)

(72) Inventors: Michael Hirmer, Wiesent (DE); Claus Jaeger, Regensburg (DE); Maria Liebl, Regensburg (DE); Stefan Strüwing, Tegerheim (DE); Dirk Sossenheimer, Lippstadt (DE)

(21) Appl. No.: 15/560,693

(22) PCT Filed: Mar. 23, 2016
(86) PCT No.: PCT/EP2016/056409

§ 371 Date: Sep. 22, 2017

(30) Foreign Application Priority Data
Mar. 23, 2015 (DE) 10 2015 104 312.2

(51) Int. Cl. A61B 5/024; A61B 5/1455; A61B 5/00

(52) U.S. Cl. CPC A61B 5/02427; A61B 5/14552; H01S 5/1082; A61B 5/6826; A61B 5/7203

(57) ABSTRACT

A sensor that senses a biometric function includes at least one transmitter configured to transmit electromagnetic radiation in an emission direction, including at least one receiver configured to receive electromagnetic radiation in a receiving direction, wherein the transmitter and the receiver are configured such that the emission direction of the transmitter is inclined away from the receiving direction of the receiver by a defined angle, wherein the angle is 1° to 60°.

### TECHNICAL FIELD

This disclosure relates to a sensor that senses a biometric function, and to a method of sensing a biometric function.

### BACKGROUND

Photoplethysmographs may be used to measure a pulse rate, for example, at a wrist or at a finger of a human being on the basis of electromagnetic radiation with the aid of a transmitter and a receiver. Known sensors have a poor signal-to-noise ratio.

It could therefore be helpful to provide an improved sensor that senses a biometric function, in particular senses a pulse of a human being or the blood oxygen content of a human being.

### SUMMARY

We provide a sensor that senses a biometric function including at least one transmitter configured to transmit electromagnetic radiation in an emission direction, including at least one receiver configured to receive electromagnetic radiation in a receiving direction, wherein the transmitter and the receiver are configured such that the emission direction of the transmitter is inclined away from the receiving direction of the receiver by a defined angle, wherein the angle is 1° to 60°.

We also provide a method of sensing a biometric function including transmitting electromagnetic radiation in an emission direction by a transmitter, and receiving reflected electromagnetic radiation in a receiving direction by a receiver, wherein the transmitter and the receiver are configured such that the emission direction of the transmitter is inclined away from the receiving direction of the receiver by a defined angle of 1° to 60°.

We further provide a sensor of sensing a biometric function including at least one transmitter configured to transmit electromagnetic radiation in an emission direction, including at least one receiver configured to receive electromagnetic radiation in a receiving direction, wherein the transmitter and the receiver are configured such that the emission direction of the transmitter is inclined away from the receiving direction of the receiver by a defined angle of 1° to 60°, wherein the transmitter including a first reflector, the first reflector defines the emission direction, the receiver including a second reflector, the second reflector defines a receiving direction, the first reflector has a parabolic shape, the transmitter is arranged at a focus of the parabolic shape of the first reflector, the second reflector has a parabolic shape, and the receiver is arranged at a focus of the parabolic shape of the second reflector.

### BRIEF DESCRIPTION OF THE DRAWINGS

FIG. 1 illustrates a schematic illustration of a sensor.

FIG. 2 illustrates a schematic illustration of a transmitter and of a receiver of a sensor.

FIG. 3 illustrates a perspective plan view of a sensor.

FIG. 4 illustrates a schematic cross section through the sensor from FIG. 3.

### LIST OF REFERENCE SIGNS

1 Sensor
2 Transmitter
3 Receiver
4 Carrier
6 Housing
7 Cover
8 Wall
9 Circuit Board
10 Finger
11 Bone
12 Evaluation Unit
13 Electromagnetic Radiation
14 Reflected Radiation
15 Artery
16 First Reflector
17 Second Reflector
18 First Lens
19 Second Lens
20 Material
21 Emission Direction
22 Receiving Direction
23 Angle
24 Emission Angle Range
25 Receiving Angle Range
31 First Recess
32 Second Recess

### DETAILED DESCRIPTION

One advantage of our sensor is that the signal-to-noise ratio is improved. This is achieved by the fact that an emission direction of the sensor is arranged in a manner inclined away relative to a receiving direction of the receiver by a predefined angle range, in particular by an angle of 1 degree to 60 degrees. Our experiments have shown that an improved signal-to-noise ratio may be achieved with the aid of this arrangement. For example, at a transmitter-receiver distance of 3-5 mm, it is possible to achieve good results at an angle range of 20 degrees to 40 degrees, in particular at an angle range of around 30 degrees.

The sensor may comprise one or a plurality of transmitters comprising an emission angle of at most 40 degrees, in particular at most 35 degrees or less. A small emission angle range additionally increases the signal-to-noise ratio on the part of the receiver. Ideally, the light is emitted parallel to the optical axis of the transmitter.

The transmitter(s) may comprise a reflector, wherein the reflector defines the emission direction and/or the emission angle range. Use of a reflector makes it possible to define a desired emission direction and/or a desired emission angle range in a simple and cost-effective manner.

The receiver(s) may comprise a reflector, wherein the reflector defines a receiving direction and/or a receiving angle range of the receiver.

Our experiments have shown that a reflector comprising at least partly a parabolic shape brings about a further improvement of the sensor. A reflector of parabolic shape may be advantageous both for the transmitter and the receiver.

The transmitter and/or the receiver may comprise a lens suitable to define an emission direction and/or a receiving direction or an emission angle range or a receiving angle range. Alignment of the radiation may be achieved by the use of a prism.

The transmitter and the receiver may be arranged alongside one another on one side of a carrier, that is to say accommodated in one component.

The above-described properties, features and advantages and the way in which they are achieved will become clearer and more clearly understood in association with the following description of the examples explained in greater detail in association with the drawings.

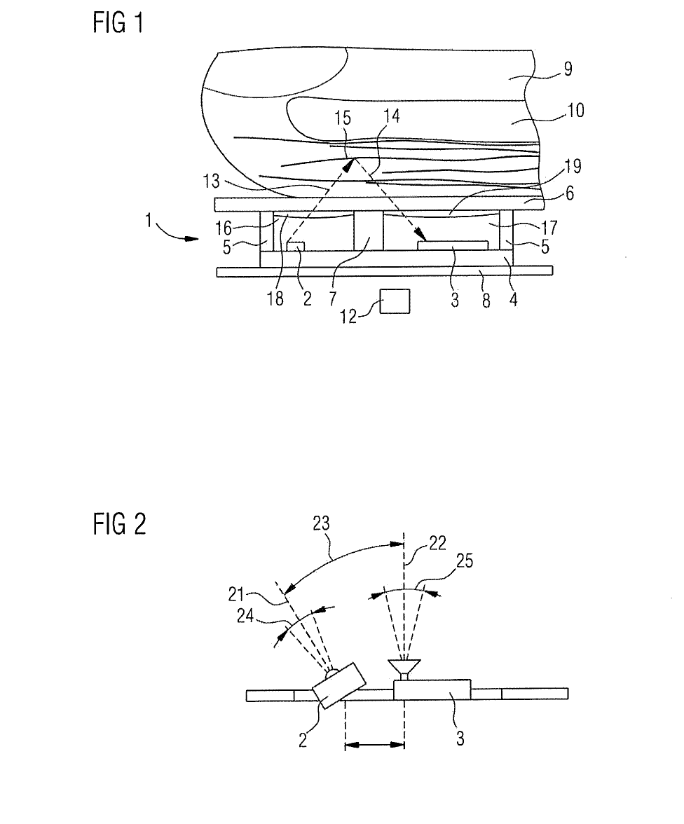

FIG. 1 shows, in a schematic illustration, a cross section through a sensor 1, wherein the sensor 1 comprises a transmitter 2 and a receiver 3. The transmitter 2 is configured to generate electromagnetic radiation 13 and emit it in a predefined emission direction and/or in a predefined emission angle range. The transmitter 2 may be configured, for example, as a light-emitting diode or as a laser diode. By way of example, the radiation output by the transmitter 2 may constitute green light. Depending on the example chosen, the light may also comprise other wavelengths.

The receiver 3 is configured to receive reflected electromagnetic radiation 14 in a predefined receiving direction and/or in a predefined receiving angle range. The receiver 3 is configured, for example, as a photodiode that converts incident light into an electrical signal. An evaluation unit 12 may be provided to evaluate the electrical signal, the evaluation unit being arranged on the sensor 1 and electrically connected to the receiver 3.

A basic principle of the sensor 1 consists of the electromagnetic radiation 13 of the transmitter 2 being emitted in the direction of a measurement object, for example, a finger 9. The finger 9 comprises skin, bones 10, arteries 15, veins and muscles. The electromagnetic radiation 13 penetrates into the skin of the finger 9 and is scattered and (partly) absorbed by body cells. In this case, the optical properties (scattering/absorption) of blood differ from those of the surrounding body cells. The returned light is modulated by volumetric expansion of the artery during the heartbeat.

At the same time, unmodulated electromagnetic radiation is scattered in the direction of the receiver 3 by other parts of the finger that do not pulsate. The modulated scattered radiation 14 brings about a corresponding modulation of the electrical signal of the receiver 3. A heart rate can thus be detected on the basis of the modulation.

A main proportion of the unmodulated reflected radiation is caused by lower skin and vein layers. An increase in the useful signal, that is to say an increase in the modulated reflected radiation 14, is achieved with the aid of the sensor.

In the illustrated example, the transmitter 2 and the receiver 3 are arranged on a common carrier 4. The carrier 4 in turn is arranged on a circuit board 8. In addition, a wall 7 is provided between the transmitter 2 and the receiver 3, which wall prevents direct irradiation of the receiver 3 by the transmitter 2. Furthermore, the transmitter 2 and the receiver 3 are surrounded by a housing 5 in a ring-shaped fashion. In addition, a cover 6 is applied on the housing 5 and the wall 7. The cover 6 is transmissive to the electromagnetic radiation 13 and the reflected electromagnetic radiation 14. Depending on the example chosen, the cover 6 may consist of glass, for example. For a measurement, the finger 9 bears e.g. directly on the cover 6. A defined distance between the transmitter 2 and the finger 9 and between the receiver 3 and the finger 9 is defined as a result.

Our experiments have shown that an increase in the useful signal may be achieved by an emission direction of the transmitter 2 being arranged in a manner inclined away from the emission direction of the receiver by a predefined angle relative to a receiving direction of the receiver 3. The angle may be 1 degree to 60 degrees, in particular 20 degrees to 40 degrees. In addition, the angle may be around 30 degrees.

FIG. 2 shows the transmitter 2 with an emission direction 21 in a schematic illustration. In addition, the receiver 3 with a receiving direction 22 is illustrated schematically. In the example illustrated, the emission direction 21 is arranged in a manner inclined away from the receiving direction 22 by an angle 23 of 30 degrees. As already explained, instead of the angle 23 of 30 degrees, some other angle range of 1 degree to 60 degrees, in particular 20 degrees to 40 degrees, may also be provided. The emission direction 21 defines a center of an emission angle range 24. The receiving direction 22 defines a center of a receiving angle range 25. The emission angle range 24 defines the angle range in which a significantly intensity of the electromagnetic radiation 13 is emitted.

By way of example, a value greater than 10% of the maximum intensity may be assumed as a significant intensity. Our experiments have shown that the useful signal is increased further if the emission angle range of the transmitter 2 is less than 40 degrees, in particular less than 35 degrees, or even less. With increasing parallel emission of the electromagnetic wave 13, i.e. with a decreasing emission angle from the transmitter 2, an increasing rise in the intensity of the useful signal is established on the part of the receiver 3.

Both for a precise definition of the emission direction 21 of the transmitter 2 and for a precise definition of the receiving direction 22 of the receiver 3, it is possible to use both reflectors 16, 17 and lenses 18, 19 (FIG. 1). Depending on the example chosen, either a lens or a reflector may be provided to define an emission direction and/or an emission angle range. In addition, both a reflector and a lens may be provided to define a receiving direction and/or a receiving angle range of the receiver. Depending on the example chosen, the lens may be configured, for example, as a prism.

In the configuration of the reflectors 16, 17 we found that a parabolic shape both for the transmitter 2 and the receiver 3 brings an increase in the useful signal. As parallel emission of the electromagnetic radiation 13 as possible from the transmitter 2 may be brought about with the aid of the parabolic shape for the reflector. Furthermore, an increase in the useful signal may be achieved with the aid of a parabolic reflector 17 at the receiver 3. The parabolic shape of the reflector enables narrow-angled beam shaping, ideally parallel beam shaping.

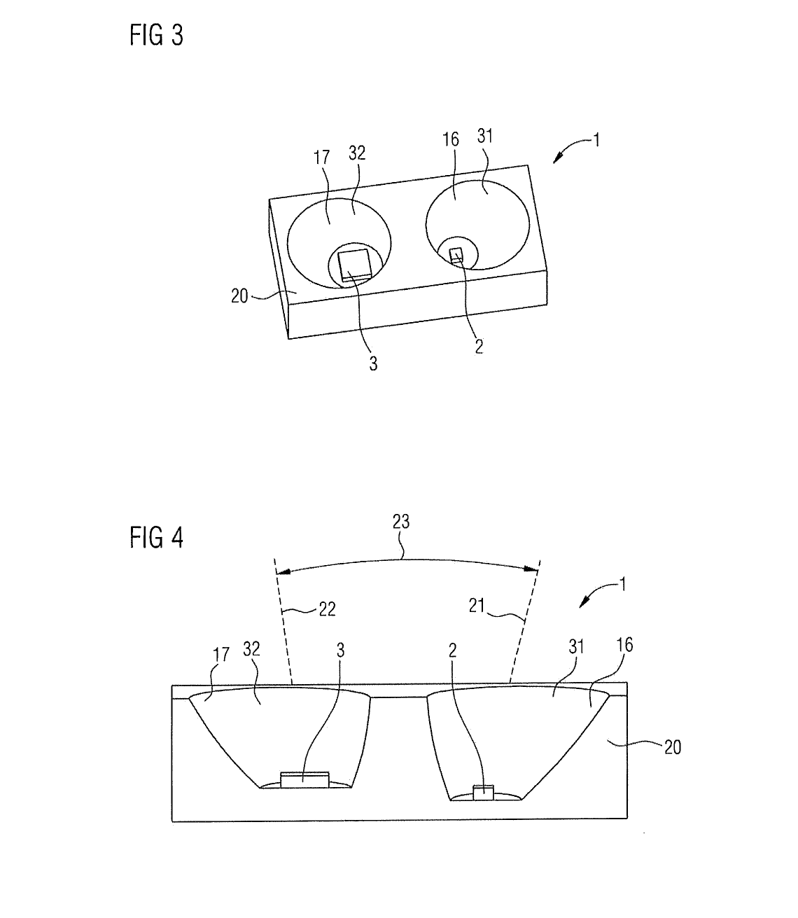

FIG. 3 shows one example of a sensor 1, wherein a transmitter 2 and a receiver 3 are provided. The transmitter 3 is arranged in a first recess 31 of a material 20. The receiver 3 is arranged in a second recess 32 of the material 20. In the example illustrated, the sidewalls of the first and second recesses 31, 32 are configured as reflectors 16, 17 with a corresponding coating, in particular with a corresponding metallic coating. In addition, the walls of the first and second recesses 31, 32 comprise a parabolic shape in the illustrated example.

FIG. 4 shows a cross section through the arrangement from FIG. 3. Consequently, the wall of the first recess 31 is configured in the form of a first reflector 16 comprising a parabolic shape. Furthermore, the wall of the second recess 32 is configured in the form of a second reflector 17 comprising the shape of a parabolic reflector. The material 20 may comprise a plastics material, for example. In addition, the sensor 1 may be produced, for example, with the aid of a Midled technology.

Furthermore, FIG. 4 illustrates the emission direction 21 of the first reflector 16 and the receiving direction 22 of the second reflector 17. The emission direction 21 and the receiving direction 22 are arranged in a manner inclined away from one another by a predefined angle 23. As already explained, the predefined angle may be 1 degree to 60 degrees, in particular 20 degrees to 40 degrees, for example, around 30 degrees. In this example, too, the emission direction and/or the receiving direction are/is defined by a center, i.e. a center axis, of an emission range and by a center, i.e. a center axis, of a receiving range. Depending on the example chosen, it is possible to dispense with the second reflector 17 at the receiver 3.

In addition, depending on the example chosen, the sensor, constituting a photoplethysmograph, may be configured as a combined component, wherein the transmitter and the receiver are arranged in the same component. In addition, the sensor may be constructed from a plurality of discrete components.

The definition of the emission direction and/or of the receiving direction may be achieved by a corresponding tilted arrangement of the reflector relative to a surface of the carrier 4, in particular a chip surface. In addition, the corresponding alignment of the emission direction and/or the receiving direction may be realized by a correspondingly tilted lens. In addition, a transmitter or a receiver may be arranged in a manner offset relative to a lens or a reflector. Furthermore, a prism or a prism array may be provided above the transmitter and/or the receiver 3 for the corresponding definition of the emission direction and/or of the receiving direction. In addition, the emission angle range and the emission direction of the transmitter and/or the receiving angle range and the receiving direction of the receiver may be defined by corresponding reflectors.

Furthermore, our experiments have shown that the greater the wavelength of the electromagnetic radiation 13 emitted by the transmitter 2, the smaller the angle 23 may be to achieve an increase of the useful signal, in particular control of the useful signal.

With the use of a reflector in the form of a parabolic reflector, the receiver and/or the transmitter are/is preferably arranged at the focus of the parabolic reflector.

Although our sensors and methods have been more specifically illustrated and described in detail by the preferred examples, nevertheless this disclosure is not restricted by the examples disclosed and other variations can be derived therefrom by those skilled in the art, without departing from the scope of protection of the appended claims.

This application claims priority of DE 10 2015 104 312.2, the subject matter of which is incorporated herein by reference.

1-9. (canceled)

10. A sensor that senses a biometric function comprising at least one transmitter configured to transmit electromagnetic radiation in an emission direction, comprising at least one receiver configured to receive electromagnetic radiation in a receiving direction, wherein the transmitter and the receiver are configured such that the emission direction of the transmitter is inclined away from the receiving direction of the receiver by a defined angle, wherein the angle is 1° to 60°.

11. The sensor according to claim 10, wherein the transmitter comprises an emission angle of 40° or less.

12. The sensor according to claim 10, wherein the transmitter comprises a reflector, the reflector defining the emission direction and/or the emission angle range.

13. The sensor according to claim 10, wherein the receiver comprises a reflector, the reflector defining a receiving direction and/or a receiving angle range.

14. The sensor according to claim 12, wherein the reflector at least partly comprises a parabolic shape, and the transmitter is arranged at a focus of the parabolic shape.

15. The sensor according to claim 13, wherein the reflector at least partly comprises a parabolic shape, and the receiver is arranged at a focus of the parabolic shape.

16. The sensor according to claim 10, wherein the transmitter and/or the receiver comprise(s) a lens for beam guiding.

17. The sensor according to claim 16, wherein the lens is configured as a prism.

18. The sensor according to claim 10, wherein the transmitter and the receiver are arranged alongside one another on one side of a carrier.

19. The sensor according to claim 10, wherein the emission direction is defined by a center of an emission range.

20. The sensor according to claim 10, wherein the receiving direction is defined by a center of a receiving range.

21. The sensor according to claim 10, wherein the transmitter and the receiver are arranged on a common carrier, and the emission direction and/or the receiving direction are achieved by a tilted arrangement of a reflector relative to a surface of the carrier.

22. The sensor according to claim 10, wherein the transmitter and the receiver are arranged on a common carrier, and the emission direction and/or the receiving direction are achieved by a tilted arrangement of a lens.

23. A method of sensing a biometric function comprising transmitting electromagnetic radiation in an emission direction by a transmitter, and receiving reflected electromagnetic radiation in a receiving direction by a receiver, wherein the transmitter and the receiver are configured such that the emission direction of the transmitter is inclined away from the receiving direction of the receiver by a defined angle of 1° to 60°.

24. A sensor of sensing a biometric function comprising at least one transmitter configured to transmit electromagnetic radiation in an emission direction, comprising at least one receiver configured to receive electromagnetic radiation in a receiving direction, wherein the transmitter and the receiver are configured such that the emission direction of the transmitter is inclined away from the receiving direction of the receiver by a defined angle of 1° to 60°,
wherein the transmitter comprises a first reflector, the first reflector defines the emission direction, the receiver comprises a second reflector, the second reflector defines a receiving direction,
the first reflector has a parabolic shape, the transmitter is arranged at a focus of the parabolic shape of the first reflector, and
the second reflector has a parabolic shape, and the receiver is arranged at a focus of the parabolic shape of the second reflector.
